(19) United States
(12) Patent Application Publication
(10) Pub. No.: US 2018/0374581 A1
(43) Pub. Date: Dec. 27, 2018

(54) HOSPITALIZATION ADMISSION RISK ASSESSMENT TOOL AND USES THEREOF

(71) Applicant: ALLYALIGN HEALTH, INC., Glen Allen, VA (US)

(72) Inventors: Robert Alan BERRINGER, Henrico, VA (US); Amy Elizabeth KASZAK, The Woodlands, TX (US); Tena Mayo KELLY, Cayce, SC (US); Will SAUNDERS, (US)

(21) Appl. No.: 16/063,005

(22) PCT Filed: Dec. 15, 2016
(86) PCT No.: PCT/US2016/067025

§ 371 Date: Jun. 15, 2018

Related U.S. Application Data
(60) Provisional application No. 62/267,801, filed on Dec. 15, 2015.

(51) Int. Cl. G16H 50/30; G06Q 50/22

(52) U.S. Cl. CPC G16H 50/30; G06F 19/00; G06Q 50/22

(57) ABSTRACT

A secure and automated computerized system providing a computerized program product and service method for integrating disparate data sources and assessing risk of hospital admission of an individual is disclosed. Individuals who are long-term residents of a nursing facility may be stratified into high, medium, or low risk groups, and the information used by health care service providers. The system also includes methods for providing an individualized resident “continuum of care” plan for a particular resident. A unique set of covariate elements for use in the automated computerized method and system is also provided.

### CROSS-REFERENCE TO RELATED APPLICATIONS

The present application claims priority to U.S. Provisional Patent Application 62/267,801, filed Dec. 15, 2015, the contents of which is specifically incorporated herein in its entirety.

### BACKGROUND

### Technical Field

The present invention relates to the field of risk assessment systems, as a system/method for assessing risk of admission to a hospital of a resident in a nursing facility.

### Description of Related Art

Current technologies and assessments focus on specific areas (e.g., mobility, cognition), diseases (e.g., dementia, diabetes), patient care areas (e.g., skilled nursing facilities) or limited data sets (e.g., health risk assessment). None incorporate assessment of multiple areas, disease, patient care areas and data sets.

Hospitalizations are disrupting to elderly individuals and puts them at greater risk for complications and infections. They negatively impact the medical, emotional, and psychological state of patients and their caregivers and cost Medicare billions of dollars. Preventing these events whenever possible is always beneficial to patients and has been identified by policymakers and providers as an opportunity to reduce overall health care system costs through improvements in quality.

Across all payers, there were 3.3 million hospital readmissions in 2011. Medicare and Medicaid accounted for 55.9% and 20.6%, respectively, of the number of readmissions and 58.2% and 18.4%, respectively of overall costs. Dual eligible beneficiaries account for a disproportionate share of Medicare spending with inpatient hospitalizations being a major driver. These beneficiaries are almost twice as likely to be hospitalized as a non-dual eligible beneficiary and associated costs are also higher than other Medicare beneficiaries. Of all hospitalizations for dual eligible members, 26% have been identified as potentially avoidable. Medicaid nursing facilities or Medicare skilled nursing facilities have the highest readmission rates compared to dual eligible living in the community or in a HCBS waiver.

Five conditions account for almost 80% of potentially avoidable hospitalizations among all dual eligible beneficiaries. Pneumonia was the leading cause of all potentially avoidable readmissions with urinary tract infections, congestive heart failure, dehydration, and falls/trauma collectively accounting for 78% and 77%, respectively, for total potentially avoidable readmissions. For dual eligible beneficiaries residing in an institution, pneumonia accounted for nearly 30% of potentially avoidable hospitalizations while urinary tract infections and dehydration were also leading causes. Falls/trauma accounted for higher proportion of potentially avoidable hospitalizations for dual eligible livings in a nursing home. Xing, et al. reported that more than half of residents were hospitalized at least once in the year prior to death and that almost half of these admissions were potentially avoidable.

Section 3025 of the Affordable Care Act added section 1886(q) to the Social Security Act established the Hospital Readmissions Reduction Program, which requires CMS to reduce payments to IPPS hospitals with excess readmissions, effective for discharges and began on Oct. 1, 2012. H.R. 4302, the Protecting Access to Medicare Act of 2014, is a value-based purchasing (VBP) program for skilled nursing facilities (SNFs). This program establishes a hospital readmissions reduction program for these providers, encouraging SNFs to address potentially avoidable readmissions by establishing an incentive pool for high performers. The Congressional Budget Office scored the program to save Medicare $2 billion over the next 10 years.

Currently, there are multiple technologies and solutions such as non-contact monitoring solutions, care transitions software, quality improvement programs, and disease management solutions that focus on this issue. However, they primarily focus on hospital readmissions in the acute care and post-acute care settings, and not hospitalizations in the nursing facility long term care setting or as among a geriatric population of patients.

Despite the above and other approaches, the medical arts remain in need of systems and methods for more effectively managing the growing population of persons in long term care nursing facility, especially among geriatric patients, so as to reduce the incidence of hospital admission factors that contribute to repeated hospitalizations and the consequences associated with patient admission to a hospital.

### SUMMARY

The present invention, in a general and overall sense, relates to a method and system for assessing risk of hospital admission and/or readmission for an individual, such as an individual that is a resident of a long or short term case facility, such as a nursing home. From this assessment of relative risk (High, Medium or Low), a treatment plan, management/visitation schedule, or other protocol or intervention appropriate for the individual may be created and implemented. The method and system is designed to reduce the risk of hospital admissions and/or readmissions, and to enhance the health condition of the individual, and/or to avoid the deterioration of the health condition of the individual so as to avoid the risk of hospitalization and/or recurrent hospitalization, of an individual and/or short and/or long term chronic or acute care facility resident, such as a patient.

In particular embodiments, the method and system may be described as a multidisciplinary methodology for the design and delivery of services to a specific population of individuals. For example, one specific population of individuals may comprise individuals determined to be at a higher risk of hospital admission than the general population. Individuals at a higher risk of hospital admission include residents of a nursing home facility, who are documented to have multiple comorbidities, are eligible for both Medicare and Medicaid (i.e., dually eligible), have impaired cognition, and have a documented history of one or more (multiple) hospitalizations within the immediately preceding year. Another characteristic of a population of persons considered to be at higher risk of hospitalization are individuals who are currently enrolled in hospice care. Additionally, the number of hospitalizations in a preceding year from evaluation of a particular individual, and specific events that provide health related information of a particular individual (e.g., lab results, length of stay, non-elective admission status) are considered in calculating relative risk of future hospital admissions and/or hospital readmission following a single hospitalization episode.

Age alone, nor any other specific individual factor described here, are not to be considered a limiting factor in the application of the present invention, as the method and system may also be applied to younger individuals having other extenuating health circumstances that require daily health care attention from a skilled health care provider, even for attention to a chronic health care episode or acute health care episode.

As used in the present invention, the term “long-teim” resident of a skilled nursing facility is defined as a person who has been residing in a nursing facility for at least 100 consecutive days, and who requires daily care by a health care professional, such as a physician, physician's assistant, nurse, nurse's assistant, or other daily health care giver, in performing routine, day-to-day tasks. Multiple factors, including independent performance of activities of daily living, medical nursing needs, clinical complexity of a persons' condition, cognition, behavior, physical environment, living area conditions, functional status, financial status, and caregiver support, for example, are to be considered in the evaluation of an individual being eligible for long-term care nursing facility services.

The methodology and system is designed to provide information to a specific user (for example, a health care provider, nurse practitioner, clinician, hospital administrator, physician assistant, geriatric facility worker, etc.) that is specific to the needs of that specific user and/or their health care organization, such as a nursing home, hospital, hospital management organization, health care management organization, insurance company, or other organization where health care management of a person/persons is of interest. Such care may be of interest to the organization where, for example, more efficient, cost effective, and patient-centric care may be provided to reduce the probability of hospital admission and/or hospital readmission, and increase the probability that the person/persons will successfully remain in a resident facility situation, such as a nursing home.

The methodology and system uses data on the medical, psychological, social, and functional capabilities and needs of particular person/persons of interest. The collected data is then used to develop person-centered treatment and long-term follow-up plans that address medical, behavioral, necessary long term care and support systems, and individual social needs of the individual.

The method and system of the invention is described in some embodiments as comprising a “Health Risk Assessment Tool” (HRAT) and a “Hospital Admission Risk” (HAR) Index (See FIG. 1). The HRAT is a multidisciplinary comprehensive individual assessment system and methodology that comprises a selected, standardized data set that can be used to assess hospitalization risk and in providing a continuum of care for an individual in need thereof. The method and system is designed to create more efficient and accurate treatment alternatives for a particular individual.

In some embodiments, the method provides a service whereby a facility may monitor and manage the facility population. For example, for a long-term nursing facility administrator, the administrator is provided a tool whereby care of facility residents may be improved and hospitalization incidence reduced. For example, a long-term living facility manager having a resident population who are at least 60 to 65 years old, and who have had at least one prior hospitalization admission incidence, can be informed of the relative risk that a particular resident may be admitted or readmitted to a hospital, and may in turn, may then make appropriate modifications in the resident's care to reduce the relative risk that the resident and/or individual will be readmitted to a hospital within a relatively short, defined period of time. The ability to assess this risk and act accordingly to reduce probability that an individual will be readmitted to a hospital is expected to significantly decrease costs to hospitals and/or individual care facilities. This will be accomplished by modification of current treatment plans for an individual and/or considering a treatment plan for the individual that accommodates and thus reduces the probability that the individual will experience an event that would increase the probability of a subsequent hospitalization.

In some embodiments, the system and methodology utilizes an individual resident's collected data on a defined and select set of uniquely combined covariate factors. The data collected on the covariant factors is used to calculate the individual resident's Risk Score (Individual Risk Score). The Individual Risk Score is then used to stratify the individual in one of three Risk Groups, of high risk group, a medium risk group, or low risk group. The covariate factors as described in relation to the present invention includes factors of the individual's medical, psychosocial and functional capabilities, and limitations, that render the individual in need of daily trained heath care attention. From the individuals “Risk Class” (high, medium or low), an treatment plan tailored to the needs of the individual is developed that is designed to provide an appropriate continuum of care that will reduce the probability that the individual will be admitted and/or readmitted to a hospital, as well as to improve the overall health condition of the individual.

For example, the individuals Risk Score, and identified Risk Class that the Risk Score places him/he into, may be used to develop a tailored treatment plan, to arrange and/or recommend other services for the individual (e.g., dietary, therapy, specialists), define frequency of follow up (e.g., face-to-face, phone, or computer assisted electronic visit), assign clinical protocols (e.g., antibiotic stewardship, hospital admission prevention, disease management, or other chronic care improvement), identify short-term and long-term screening schedules, modification and/or change to the type of care facility or care program that the individual will be placed in, among other things. Ultimately, the method and system will provide the best care options for the individual, while at the same time making the most efficient use of health care resources for the nursing care facility.

In some embodiments, the Risk Score of an individual may be described as being calculated using a proprietary algorithm that incorporates data collected for a proprietary set of 22 or more selected covariate parameters. The individual Risk Score is then used as part of a Health Risk Assessment (HRA) Tool. The HRA Tool also employs a proprietary algorithm that provides a self-contained, step-by-step set of actions and/or calculations utilizing a series of operations to be performed to provide a treatment/management planning tool for an individual, as well as a management tool that may be used by a nursing facility/long term residence facility.

The Risk Score of a particular individual is an evidence-based scoring methodology. The methodology includes the assessment of a proprietary set of covariant parameters, and particularly, a set of 22 or more selected covariate parameters. In a particular embodiment, the set of covariates comprises 22 data points. Reference is made to Table 1, which includes what is included in the HRA Tool, from which an individual Risk Score calculation is derived (#4—Most vulnerable beneficiary risk index—Hospital Admission Risk Index). The covariate factors have been identified by the present inventors to be statistically predictive of the individual's health risk, especially heath risk for hospital admission and/or readmission. The method is particularly predictive of hospitalization and/or re-hospitalization risk among long-term residents of a nursing facility.

A “covariate,” as used in the description of the present invention, is intended to describe a selected characteristic, such as a clinical, demographic feature and/or condition of a resident. Calculations using these individual covariates provide a means for stratifying a specific resident's risk, relative to a given population of like-residents, for hospitalization and/or re-hospitalization within a defined period of time following an initial hospitalization of that resident. (such as a defined period of within a 12 month period immediately following an initial hospitalization admission).

According to some embodiments of the invention, a Risk Score of an individual may be calculated using a computer implemented system. The computer system will comprise, for example, an input station having a display unit, the station being suitable for entry or information by a user, a memory suitable for facilitating the operation and execution of a series of programmable operations (such programmable operations as may be specified by an appropriate software system (code)), and a central processing unit. The Risk Score of an individual may also be calculated using a computer implemented system comprising an input station having a display unit, a memory suitable for facilitating the operation and execution of a series of programmable operations (such as programmable operations as may be specified by an appropriate software system (code)) and a central processing unit. Accordingly, the Risk Score calculated for an individual is used to electronically assign the individual into a Risk Group. Based on the individual's Risk Score, the individual is categorized into a Risk Group. This analysis involves the stratification of the individual into a high (Risk Score of greater than about 2,000 points), medium (Risk Score of about 1,100 to less than about 2,000 points), or low (Risk Score of not greater than about 1,100 points) Risk Group. Those in the high Risk Group being identified as at a higher risk of hospital admission and/or readmission than those individuals in a low or medium Risk Group.

It will be required that access to the data items and data sets will be restricted to certain users for privacy, HIPAA, FERPA, and other reasons. In order to apply these restrictions, an information management system will be part of the present methods and systems, and will determine the identity of the user requesting access. This may be done in many ways, but in some embodiments, will be done by physically measuring a unique quality of the uses of requesting information from the user, or by using a specific password for each authorized user that provides the user either a defined scope of access or more complete scope of access to the system, depending on the authorization level of the user. A password system for access should never be written down or embedded into a login script and should always be interactive. Accordingly, in a password system, a user's identity will be determined through an extensive question and answer session. The responses to certain personal or institutional questions will identify an authorized user with high accuracy.

Data collected in the BRAT and Admission Risk Index, and data on enrollment, pharmacy claims history, medical claims history, and nursing facility data, is used to develop a treatment plan and/or a long-term follow up care plan for the individual. These individual identifiers provided according to the present invention will impact the clinical outcomes of the individual, such as relative risk of subsequent hospitalizations, ED visits, length of stay projections, and suitability of quality of care.

FIG. 1 presents a flow chart that illustrates the system/method, that comprises the HRAT and Admission Risk Index process. The system/method presents a tool to create an initial individual overall health care assessment, individual health care planning regimen and individual health care follow up plan for an individual, and functions as a tool to be used to improve the individuals' future health assessment relative to an initial health assessment.

The present method and system, termed the Align36™, and that incorporates the HRAT and Hospital Admission Risk Index described here, provides many advantages over current practices in managing and evaluating an individual by providing a customized and more tailored and appropriate health care plan for the individual. By way of example, some of the advantages of the present methods and systems include:
Integration of disparate data sources (e.g., enrollment, medical claims, pharmacy claims, MDS, HRA),
Application of evidence-based algorithms using table-driven rules engine
Automation of a long teen care patient risk score for hospital admission

In yet another embodiment, a nursing home resident data analysis system is provided. In one embodiment, the system comprises a computer having a memory, a central processing unit and a display. The system is further defined as comprising a means for configuring said memory to store and perform a set of defined functions on a defined set of covariant elements as defined in Table 2, a means for providing said central processing unit with data input into the memory and a means configured to relay a defined set of covariant elements into to the central processing unit. In some embodiments, the display is a computer screen provided at an input portal. In preferred embodiments the system provides for a step wherein the computer system is provided with a security system, preventing access to any user without an appropriate password or proper screening mechanism.

These and other advantages will be appreciated by those of skill in the art in view of the present disclosure.

### BRIEF DESCRIPTION OF THE DRAWINGS

The accompanying drawings illustrate a number of exemplary embodiments and are a part of the Specification. Together with the following descriptions, these drawings demonstrate and explain various principles of the instant disclosure.

FIG. 1 is a flowchart depicting a hospital readmission risk assessment system for long term care facility.

FIG. 2 is a flowchart depicting a system whereby a continuum of care plan may be devised for a resident of an assisted living facility.

FIG. 3 is a computer screen shot of the dashboard for the Login page in the present web-based system.

FIG. 4 is a computer screenshot of the homepage of the present web-based system.

FIG. 5 illustrates a screen shot of a dashboard representation of the general “Patient Details” input page that includes information such as demographics, contact (e.g., patient, power of attorney), assigned providers (e.g., doctors, nurse practitioners), insurance, and social history.

FIG. 6 illustrates a screen shot of a dashboard representation of the social “Patient Details input user interface page of the present web-based system.

FIG. 7 illustrates a screen shot of the dashboard for entry of the Minimum Data Set (MDS) user interface page of the present web-based system.

FIG. 8 illustrates a screen shot of a dashboard representation of the Health Risk Assessment Tool user interface page of the present web-based system.

FIG. 9 illustrates a screen shot of a dashboard representation of the Hospital Admission Risk Index user interface page of the present web based system.

FIG. 10 illustrates a screen shot of a dashboard representation of a Medication Reconciliation user interface page of the present web-based system.

FIG. 11 illustrates a screen shot of a dashboard representation of an “Orders” “user interface of the present web-based system.

FIG. 12 illustrates a screen shot of a “Plan of Care” (“continuum of care”) user interface page of the present web-based system.

### DETAILED DESCRIPTION

As shown generally in the accompanying drawings, various embodiments of the present invention are illustrated to show the structure and relationship of the various steps of the method that comprise the systems and methods for monitoring and assessing hospitalization risk of a resident of a nursing home or facility. Common elements of the illustrated embodiments are designated with like numerals. It should be understood that the figures presented are not meant to be illustrative of actual views of any particular portion of an actual device structure and is not intended to be limiting as to any particular sequence of steps, but are intended to provide a schematic representation which may be employed to more clearly and fully depict embodiments of the invention.

The information technology (IT) system of a facility that houses or manages individuals in need of skilled nursing care or assistance, or other facility that interacts with such a facility, may use the presently designed system and methods to identify individuals at a higher or lower risk of hospitalization, as well as in identifying treatment options for an individual designed to establish an appropriate “continuum of care” so as to reduce the relative risk of the individual from admission to a hospital.

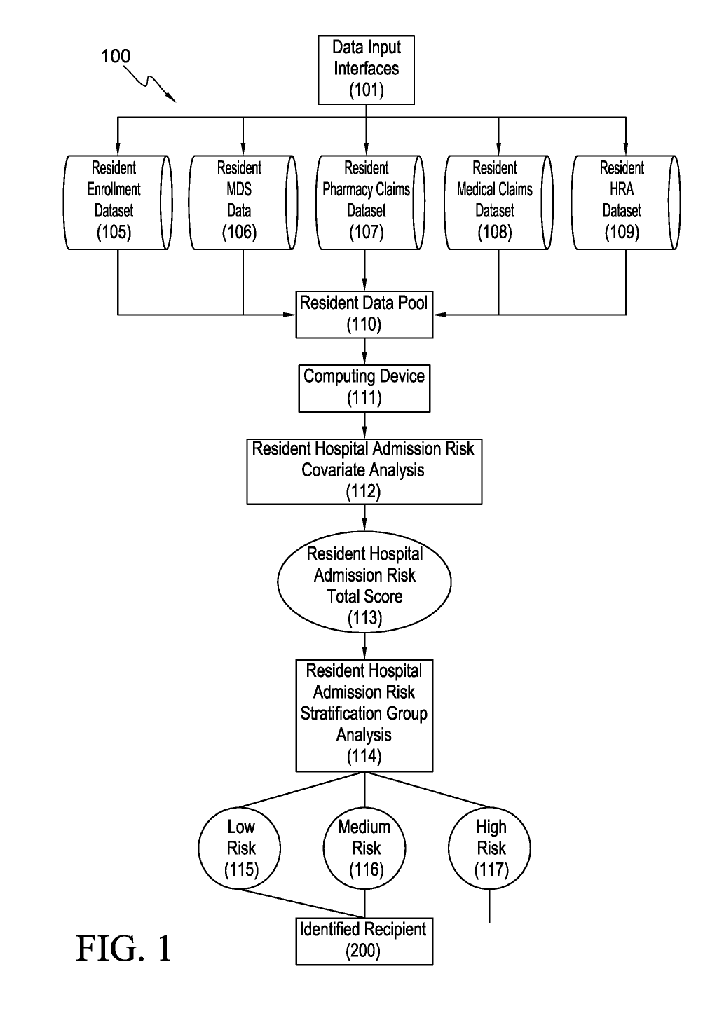

Turning now to FIG. 1, the system (100) provides for one or more Data Input Interfaces (101). The sources of data that are to be entered at a Data Input Interface (101) will in some embodiments be data that is specific for a particular individual, such as an individual who is a resident of a nursing facility, such as a nursing home. The sources of data specific for the resident include, for example, resident enrollment data (referred to as the Resident Enrollment Dataset (105)), the resident MDS Data Set (Long-term care Minimal Data Set) (106), the resident Pharmacy Claims Dataset (107), the resident Medical Claims Dataset (108) and the resident HRA Dataset (109). Other sources of data may be input into the composite of data as well. All of the resident data is provided into a computing/receiving device having the ability to store and manipulate the data, such as a computer, laptop computer, dedicated use computer, server, electronic tablet, smart phone, etc. (111). Collectively, the sum of all data collected for an individual or group of individuals is referred to as a Resident Data Pool (110).

The MDS (Long-Term Care Minimum Data Set (MDS) is a standardized, primary screening and assessment tool of health status that forms the foundation of the comprehensive assessment for all residents in a Medicare and/or Medicaid-certified long-term nursing facility.

The computing device (111) will include appropriate software that provides for the manipulation of the Resident data Pool (110) to be applied to a Resident Hospital Admission Risk Covariate Analysis (112), which is described in greater detail later in this description. The results of the manipulation and scoring of the Resident Data Pool (110), upon applying the Resident Hospital Admission Risk (HAR) Covariate Analysis (112) (employing 22 or more individual, selected covariate characteristics of the individual), results in the calculation and/or determination of an individual Resident Hospital Admission Risk Total Score (HART) (113).

The Resident Hospital Admission Risk Total Score (113) of the individual/resident is then analyzed against a reference individual/resident population of data, to determine the relative risk of the subject individual/resident being admitted to a hospital. This analysis is then used to stratify the individual/resident into a specific “Risk Group”, depending on the individual/resident's individual score. The relative risk of the individual/resident is described as Low Risk (score of 0 to 1,099) (115), Medium Risk (score of 1,100 to 2,000) (116) or High (score of 2,001 to 10,000) (117).

The results of the assessment of the individual/resident as in a Low, Medium or High risk group may then be electronically communicated to the facility, service provider, or other professional in need of such information (200). Action and/or modification of current plan of care for the individual/resident may then be made by the recipient of the individual/resident result.

In particular embodiments, the individual/resident is a geriatric individual/resident.

The Data Sets included as part of the Resident Data Pool provides in the present methods and systems a multidisciplinary diagnostic instrument that is used to collect data on the medical, psychological, social, and functional capabilities and needs of an individual/resident (elderly person).

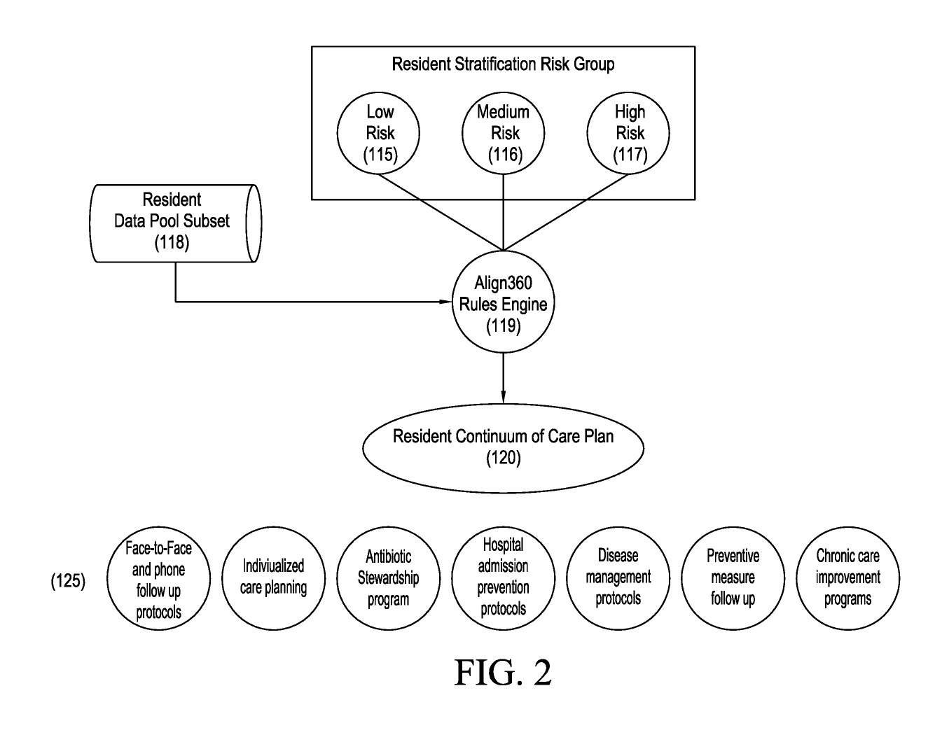

In another aspect, the method and system of the present invention may be used to provide a “Continuum of Care” plan designed to meet the needs of a specific individual/resident. In this way, a person-centered treatment and long-teun follow-up plan that address the medical, behavioral, long term care of the individual/resident, and supports the social needs of the individual/resident, may be provided. In this aspect, reference is made to FIG. 2. The method and system provides for the first identification of the “Risk Group” as described above. (Low(115), Medium (116) or High Risk(117)), and the entry of the “Risk Score” as previously determined (see above), into a data base. A Resident Data Pool Subset (118) specific for the individual/resident (see Table 4) is then entered and combined with the individual/resident “Risk Score” into a single data base. A computerized program having a series of defined parameters and selection metrics (the “Rules Engine”)(119) is then run on the single data base, and will provide a report, identifying a Resident Continuum of Care Plan (120), that will include any number of individually tailored and specific recommended elements (125), such as visitation plans (face-to-face follow-up protocols), individualized care planning), medication schedules (antibiotic stewardship program), disease management protocols (diabetes, high blood pressure, etc., preferred dietary management), preventive measure follow-up protocols, and chronic care improvement programs, among other things, for the individual/resident.

The system and method herein is referred to collectively as the Align360™ Health Risk Assessment Tool (HRAT), referred is a multidisciplinary comprehensive geriatric assessment that provides a standardized data set across a continuum of care. It is designed to collect data on the medical, psychosocial and functional capabilities, and limitation of residents of a long-term care facility (such as a resident that is assigned to a long-term care bed in a skilled nursing facility, and in need of skilled nursing services), and is useful to develop treatment plans, arrange other services (e.g., dietary, therapy, specialists), identify risk for hospitalization, to risk adjust Medicare patients by assigning a hierarchical condition category (HCC) score and ultimately make the most efficient and cost effective use of health care resources.

The software platform of the present method and system brings together and contextualises clinical information from a variety of disparate sources into a single aggregated clinical data repository and helps orchestrate care across an enterprise. The platform includes a rules engine that is a smart algorithm-based engine that embeds evidence based care protocols, analyses patient information, and generates alerts ensuring care is delivered to standards. It queries a dynamically extensible data model that collects and contextualizes data from a variety of data sources (e.g., enrollment, pharmacy claims history, medical claims history, MDS, HRA) and applies user defined rules to track clinical events, disease markers and other quality measures based upon evidence based care protocols. Pertinent notifications are internally or externally pushed to an identified recipient, such as a designated care provider or nursing home, in a secure manner. A computer program product for providing the present automated risk assessment method and system constitutes at least one aspect of the present invention, which will comprise, for example, a computer program code means suitable for collecting health care data from a plurality of data sources, including a set of the covariate elements (see Table 2), for an individual/resident; a computer program means suitable for inputting the data into a central computer database, the means being programed such that when said means is executed, it is capable of performing a health risk assessment for admission to a hospital for the resident, this central computer having a web-based application; a computer program means that upon execution is suitable for classifying the risk score for the resident as high risk, medium risk or low risk; and a computer program code means that upon execution is suitable for electronically transmitting the resident risk score classification in a secure, HIPPA compliant, format to an identified recipient.

The Medicare Modernization Act of 2003 (MMA) created Medicare Advantage (MA) which relies on the hierarchical condition category (HCC) system to formulate payments for participating managed care plans. HCC uses ICD information and matches a member's individual health risk profile with the premiums paid to the plan. ICD codes are mapped to specific HCC disease categories, which ultimately dictate the premiums paid to the Medicare Advantage plan. The risk scores consider multiple member factors such as sex, age, and diagnoses.

The Hospital Admission Risk Index (HARI) for a particular individual/resident, is determined using a number of selected data sets and steps of analysis (e.g., Resident Hospital Admission Risk Covariate Analysis (22 covariates), etc.), to provide a Resident Hospital Admission Risk Total Score (113). The Hospital Admission Risk Total Score (“HART”), is used in the calculation of an “index” (Hospital Admission Risk Index, “HARI”) value for the individual/resident, as part of the Resident Hospital Admission Risk Stratification Group Analysis (114). The HARI corresponds to the particular individual/resident's risk group (High (117), Medium (116) or Low (115) risk group. For example, an individual/resident having a HARI score of >2,000 points is identified as being at a high risk of hospital readmission. A resident having a HARI score of 1,100-2,000 points is identified as having a moderate risk of hospital readmission. A resident having a HARI score of 0 to 1,099 points is identified as having a relatively low risk of a hospital readmission.

Data collected in the present systems and methods may also be used to develop specialized treatment and long-term follow up care plans for an individual/resident. The customization of a treatment and long-term follow up care plan will impact the clinical outcome of the individual/resident, such as hospitalizations, ED visits, length of stay, and quality of care.

By way of example, a resident having a HARI score that places them in a high risk of readmission category would be advised and managed to have a treatment plan wherein a greater amount of follow-up and monitoring would be provided so as to better potentially circumvent and/or significantly reduce the probability that the resident patient would suffer a subsequent readmission to a hospital for treatment. In contrast, a resident having a HARI score that places them in a low risk of readmission category would be advised and managed to have a treatment plan wherein a lesser frequency of follow-up and monitoring would be provided, while still providing a treatment plan that is suited and/or tailored to adequately potentially circumvent and/or significantly reduce the probability that the resident patient would suffer a subsequent readmission to a hospital for treatment.

FIG. 1 illustrates the HRAT and Admission Risk Index process. The HRAT process also provides a system/method where the individual/resident's HARI (or “risk group”) may be used to create a follow-up care plan for the individual/resident (“Continuum of Care”, See FIG. 2), where the “risk group” of the individual/resident is used in creating the care plan.

### EXAMPLES

In order that the disclosure described herein may be more fully understood, the following examples are set forth. It should be understood that these examples are for illustrative purposes only and are not to be construed as limiting this invention in any manner.

### Example 1: Health Risk Assessment Tool (HRAT)

The present example describes the Health Risk Assessment Tool (HRAT). As described here, the BRAT is a multidisciplinary comprehensive geriatric assessment tool (system and/or method) that provides a standardized data set specific for an individual, and this data set is maintained and updates to the condition of the individual over time, so as to reflect changes in the individual's condition. In this manner, the HRAT is described as providing a standardized data set over a continuum of care. Objective scores generated from the evidence-based assessments included in the HRAT may be used to direct care independent of the care settings (e.g., skilled nursing facility, long tem′ care facility, home health).

In some embodiments, the HRAT assesses the 16 different areas included in Table 1, temied HRAT Components. These 16 different areas have been found to remain pertinent and relevant to the well-being of an individual across the continuum of care. Total scores are created for respective areas.

A set of inquiries are associated with each of the 16 different areas identified in Table 1. The answers obtained to the inquiries in each of the 16 different areas provide a data pool that the automated proprietary method and system of the present invention may incorporate. The data is used as part of the method and system to determine the most appropriate intervention, care plan activity, recommendation, and/or medically relevant orders for a particular individual. The data is used in the automated scoring of an individual to determine a specific intervention, care plan activity, recommendation, and/or medical order, in addition to other additional, different data in the patient's history.

The number of inquiries, or questions, that are part of each of the 16 different areas are provided in Table 1. Standard questionnaires known to those of skill in the art may be utilized for each of the specific areas recited in Table 1. For example, “Mini Mental Status Exam” as a specific area noted in the HRAT below, may be discerned with a standard questionnaire that measures cognitive impairment and is currently used in the across multiple care settings for this purpose.

However, it is to be understood that in certain areas, the specific number of questions that may be presented and collected as part of the data set may and will often times vary. Such variations are considered to be within the scope of the presently intended invention.

TABLE 1
HRAT Areas:

| | | Number of | |
|---|---|---|---|
| Area | | questions | Description |
| 1. | Questions For General | 17 | Includes demographic data and information related to |
| | Patient Information | | end of life planning. |
| 2. | Questions for Vital | 8 | Captures current patient information including height |
| | Signs | | and weight, BMI, blood pressure, and temperature. |
| 3. | Questions for Current | 8 | Captures details of current medication regimen to |
| | Medications | | include aspirin use, side effects, effectiveness, |
| | | | presence of high risk medications and potential |
| | | | harmful drug to drug interactions. |
| 4. | Allergies | 2 | Includes identification of allergies. |
| 5. | Questions for | 8 | Captures hospitalization and emergency room |
| | Hospitalizations | | utilization history details for previous 12 months. |
| 6. | Questions for Family | 17 | Captures family medical history for parents, siblings, |
| | History | | children and grandparents. |
| 7. | Questions for | 30 | Includes questions relative to evidence-based |
| | Tests/Vaccines | | medicine guidelines such as United States Preventive |
| | | | Services Task Force (USPSTF), Health Effectiveness |
| | | | Data and Information Set (HEDIS), and National |
| | | | Quality Forum (NQF). |
| 8. | Questions for Medical | 39 | Collects disease history as part of an Annual |
| | History | | Wellness Visit (AWV) used to create a hierarchical |
| | | | condition category (HCC) score used for risk |
| | | | adjustment of Medicare recipients. Includes the |
| | | | Braden Scale, that is a scale to help health |
| | | | professionals, especially nurses, assess a patient's |
| | | | risk of developing a pressure ulcer. A lower score |
| | | | indicates a lower level of functioning and higher risk |
| | | | for pressure ulcer development. A score ≥ 19 would |
| | | | indicate that the patient is at low risk with no need |
| | | | for treatment. |
| 9. | Questions for Surgical | 1 | Identifies surgical history. |
| | History | | |
| 10. | Questions for Social History | 4 | Identifies history and current use of tobacco |
| | | | products, alcohol, and illicit drugs. Includes CAGE |
| | | | assessment. |
| 11. | Questions for ADLs | 8 | Identifies patient's current level of independence |
| | | | with activities of daily living including eating, bed |
| | | | mobility, transferring, bathing and dressing. |
| 12. | Questions for Continence | 2 | Identifies presence and level of bowel and bladder |
| | | | continence. |
| 13. | Questions for Locomotion | 9 | Identifies independence as it relates to mobility and |
| | | | fall risk. |
| 14. | Questions for IADLs | 10 | Identifies independence with activities such as meal |
| | | | preparation and transportation. |
| 15. | Questions for Diet | 9 | Used to identify adults > 65 years of age who are |
| | | | malnourished or at risk of malnutrition. Score of 0-7 |
| | | | indicates malnutrition, 8-11 indicates at risk for |
| | | | malnutrition, and 12-14 indicates normal nutritional |
| | | | status. |
| 16. | Questions for Outpatient | Varies | Identifies details of current pain to include location, |
| | Assessment Pain Screening | | severity, triggering activities, methods for |
| | | | management, and patient goals for treatment. |
| 17. | Questions for Depression | 2-11 | Used to screen for depression in elderly patients. |
| | Screening | | Score ≥ 5 suggests depression. |
| | | | Tests and individual's orientation, attention, |
| 18. | Questions for Cognition | 12 | calculation, recall, language, and motor skill. It |
| | Assessment | | quantifies cognitive function and screen for cognition |
| | | | loss. |
| 19. | Questions for Medical | 10 | Allows for capture of any medical issues or |
| | conditions not addressed | | concerns not captured elsewhere. It also, identifies |
| | | | the patient's engagement with care management, |
| | | | healthcare goals, and barriers (cultural and/or |
| | | | spiritual) to attaining goals. Includes identification |
| | | | of potential or actual abuse. |

It is contemplated that other embodiments of the HRAT may include fewer or more specific “areas” for which data will be collected. Therefore, the HRAT component of the present methods and systems may include only 10, 12, 14, or 15 areas, or include, in other embodiments, 17, 18, 20, or even more areas on inquiry.

### Example 2—Hospital Admission Risk Protocol—“Covariate” Set and Individual Scoring System

The presently described Hospital Admission Risk Protocol incorporates a proprietary evidence-based risk index. The risk index incorporates a “Risk Score” value that first calculated for each individual. In a general sense, the “Risk Score” is calculated for a particular individual as the sum of cumulative “points” tallied for a particular individual based on the answers to a set of questions. As used in the description of the present invention, a subset of questions that have been identified by the present inventor to provide predictive features for determining relative risk of an individual/resident to be admitted to the hospital (and which is also used as a data set in determining an individual's “Risk Score”) is referred to here as a covariate. Table 2 provides a subset of 22 covariates. The listing is not exclusive, and additional questions may be included and/or deleted from the list in Table 2.

In some embodiments, the covariate set comprise a set of 22 questions. The number of covariates in a set may also vary, having as few as 10, 20, or 22 questions, or as many as 28, 30, 40 or more questions. In the present embodiment, the covariate set is made up of a set of 22 questions as provided in Table 2. This particular set of covariates (the terms “question” is used interchangeably with the term “covariate”) in Table 2 were identified in the present work to have a statistically significant association with higher hospital admission/readmission rates in a population of geriatric residents in a nursing facility and/or long term care setting. The answers and the point count associated with a particular answer are included in the present system's database and used in the electronic calculation and risk score assessment system.

Data answers to covariate questions, for example, “Medical Disease History” questions, will typically be input by a nursing facility clinician or clerical attendant. As shown in Table 2, in some cases, a specific “area” may comprise several specific questions, the answer to each having its own specific point value. For example, with “Medical Disease History,” the particular disease is assigned a specific point value (in a range of from 0.58 to 5.84). No “points” as assessed where there is no relevant disease history.

The 22 covariates, in some embodiments, include those items provided here in Table 2.

TABLE 2
Hospital Admission Risk Index Covariate Factors Table with Point Values

| Question | Answer | Score |
|---|---|---|
| 1. Age < 65 years | Yes or No | Y = 116 |
| 2. Gender | Male or female | Male = 106: Female = 0 |
| 3. Medicare as payor. | Yes or No | Y = 363 |
| 4. Medical Disease | Yes or No | Range Point Score for “Yes” = 30-584 |
| history? | | Anemia = 60 |
| | | Asthma = 68 |
| | | Diabetes = 30 |
| | | Heart failure = 131 |
| | | Internal bleeding = 584 |
| | | Respiratory failure = 076 |
| | | Septicemia = 058 |
| | | Viral hepatitis = 263 |
| | | No disease = 0 |
| 5. Current Cancer | Yes or No | Y = 116 |
| chemotherapy. | | |
| 6. Current radiation | Yes or No | Y = 400 |
| therapy. | | |
| 7. Current insulin. | Yes or No | Y = 116 |
| 8. Current daily pain. | Yes or No | Y = 4 |
| 9. Current complete | Yes or No | N = 218 |
| patient cognition. | | |
| 10. Currently on | Yes or No | Y = 395 |
| dialysis. | | |
| 11. Discharged from | Yes or No | Y = 416 |
| an oncology service. | | |
| 12. End stage | Yes or No | Y = 514 |
| prognosis. | | |
| 13. Current hospice | Yes or No | Y = 988 |
| care. | | |
| 14. Number of | 0, 1-5, >5 | 0 = 000 |
| hospitalizations | | 1-5 = 416 |
| in last year. | | >5 = 1041 |
| 15. Current | Yes or No | Range Point Score for “Yes” = 87-416 |
| hospitalization | | Resident admission of prior ≥5 day hospital stay. |
| | | Y = 416 |
| | | Resident admission of a non-elective type |
| | | hospitalization admission |
| | | Y = 208 |
| | | Procedures during hospitalization (any ICD-9-CM |
| | | coded procedure)? |
| | | Tracheostomy continued from hospitalization Yes, |
| | | No, NA Y = 87 |
| | | Returned to same SNF following hospitalization? |
| | | Y = 92 |
| | | Currently prescribed an IV med that was continued |
| | | from the hospital? |
| | | Y = 123 |
| | | Low Na at discharge from hospital (<135 mEq/L), |
| | | Y = 208 |
| | | Low hemoglobin at discharge from hospital (<12 |
| | | g/dL)? Y = 208 |
| 16. Currently | Yes or No | Y = 214 |
| requires ostomy care? | | |
| 17. Total bowel | Yes or No | Y = 121 |
| incontinence? | | |
| 18. Is the patient | Yes or No | Y = 309 |
| dependent for eating? | | |
| 19. Two person | Yes or No | Y = 156 |
| assistance from one | | |
| more ADLs? | | |
| 20. Current pressure | None, Stage 2, | Range score = 0-119 |
| ulcer(s)? | Stage 3, Stage | None = 0 |
| | 4, Unstageable | Stage 2 = 109 |
| | | Stage 3 = 87 |
| | | Stage 4 = 103 |
| | | Unstageable = 119 |
| 21. Current venous | Yes or No | Y = 263 |
| arterial ulcer? | | |
| 22. Current diabetes | Yes or No | Y = 96 |
| related foot ulcer | | |

A “covariate total score,” which is calculated using numerical values assigned to each “covariate” question answer (as defined in Table 2), for a particular individual (such as a nursing facility care resident), will be used to stratify the individual into one of three groups. The individual's “covariate total score” will be appropriately weighted, and used to determine a “Risk Coefficient” for a particular individual.

An individual's “Risk Coefficient” will then be normalized on a scale of 1-100. The normalized “Risk Coefficient” may then be converted to a “Risk Score.” The “Risk Score” is then used to stratifying the individual into one of three (3) risk groups, in this instance, risk of hospital admission. These three (3) groups are defined in Table 3.

TABLE 3
Stratification of Resident based on Risk Score

| | |
|---|---|
| Low Risk Group | Score 0 to about 1,099 |
| Medium Risk Group | Score about 1,100 to about 2,000 |
| High Risk Group | Score about 2,001 to about 10,000 |

The risk score for a particular individual is continuously updated as new data is loaded into the database to reflect the real-time and continuous condition of the individual. This assures that the risk score remains as accurate an assessment as possible. This also provides a means by which patient improvement or lack of improvement may be monitored and assessed. For example, if an individual's risk score decreases after having been placed on a particular treatment and/or dietary regimen, then the individual's condition may be identified as having improved. Conversely, if the individual's risk score increases, then this is an indication that the particular treatment and/or dietary regimen should be changed and/or modified, or, in extreme circumstances, halted.

### Example 3—Continuum of Care Planning and Assessment Tool

The present method and system incorporates multiple characteristics of a particular individual, including individual enrollment data, medical claims history, pharmacy claims, MDS, BRA, the incidence of specific diseases, hospital admissions data, psychosocial data, functional characteristics, and other data points that are combined in a single system to create a multidisciplinary instrument particularly valuable in the more effective management of a geriatric population. As a multi-integrated system, the present invention does not give over consideration of any one particular characteristic of an individual, and at the same time views the individual, especially the geriatric nursing home resident, as a composite patient. Insofar as this approach provides a more effective methodology for treatment of the person as a whole, it is envisioned that the overall health condition of the individual will be improved over a continuum of time, compared to approaches to geriatric resident care currently used.

As part of a continuum of care after a geriatric patient has been discharged from the hospital and is returning the a nursing facility, the methodology and system provided here may be used to select the most appropriate care setting and care regimen for the individual. For example, using the “Continuum of Care” tool, a particular resident may be directed to an SNF program, an LTC program, or a home health care program.

### Example 4—Automated System for Resident Assessment and Scoring

The present example describes the automated/computerized system for using the presently described method. Results generated using the automated system and method may be provided as a fee for service to any number of customer recipients. For example, data results generated using the present invention may be provided to a nursing home facility where a particular individual is a long-term geriatric resident, to an insurance provider, to a hospital finance services provider, a nurse health care provider, clinician, nursing facility worker or facility administrative staff, or other clinical or administrative professional, for identifying risk level for a second or subsequent hospital readmission of a resident of an nursing facility.

The system will generally include a centralized server that is configured to enable the information flow and exchange of information between an intended recipient of the information (such as a nurse practitioner, nursing facility, hospital service provider, hospital admission system, insurance provider, etc.) and a centralized server. In one embodiment, the centralized server may be configured to provide information from the data generating service to the intended recipient purchasing the service, concerning one or more individuals. For example, such data may include the assessment of risk for one or more residents of a particular nursing home facility. The service provider and/or computerized electronic service may provide notifications and/or other reports that include electronic mail systems, direct system electronic data input, electronic messaging systems and telephone systems, including land and cellular communication systems, to an indicated facility and/or recipient.

The central server will be configured to permit the input or to enable the storage of current and historical patient records, information and data associated with patients who have records with hospitals and treatment centers associated with the resident. In some embodiments, the central server can be coupled to or obtain patient data from other patient information and data sources, such as a medical record facility or records from a prior hospitalization and admission episode. In some embodiments, the medical record facility is communicatively coupled to a database of the present system, so as to facilitate the transfer of data collected by the hospital on an individual or group of individuals to the system on a continuous basis, updating a particular individuals condition in real-time.

The system and methods of the invention may include software and computer programs incorporating the process steps and instructions described above. In one embodiment, the programs incorporating the process described herein can be stored as part of a computer program product, and executed in one or more of the computers that make up the system of the present invention.

The computers can each include computer readable program code means stored on a computer readable storage medium for carrying out and executing the process steps described herein. In some embodiments, the computer readable program code is stored in a memory.

The devices and systems of the present method can be linked together in any conventional manner, including, a modem, wireless connection, hard wire connection, fiber optic or other suitable data link. Information can be made available to each of the systems and devices using a communication protocol typically sent over a communication channel or other suitable communication line or link.

The systems and devices of the embodiments disclosed herein are configured to utilize program storage devices embodying machine-readable program source code that is adapted to cause the devices to perform the method steps and processes disclosed herein automatically. The program storage devices incorporating aspects of the disclosed embodiments may be devised, made and used as a component of a machine utilizing optics, magnetic properties and/or electronics to perform the procedures and methods disclosed herein. In alternate embodiments, the program storage devices may include magnetic media, such as a diskette, disk, memory stick or computer hard drive, which is readable and executable by a computer. In other alternate embodiments, the program storage devices could include optical disks, read-only-memory (“ROM”) floppy disks and semiconductor materials and chips.

The systems and devices may also include one or more processors or processor devices for executing stored programs, and may include a data storage or memory device on its program storage device for the storage of information and data. The computer program or software incorporating the processes and method steps incorporating aspects of the disclosed embodiments may be stored in one or more computer systems or on an otherwise conventional program storage device.

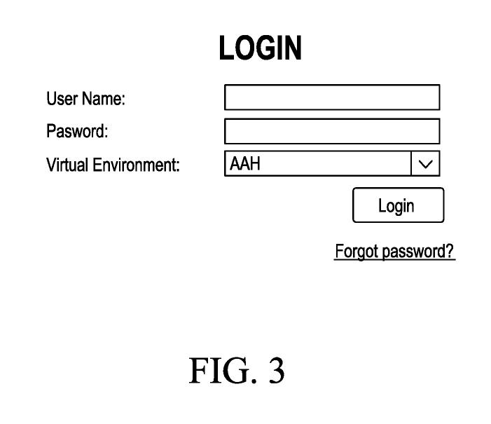

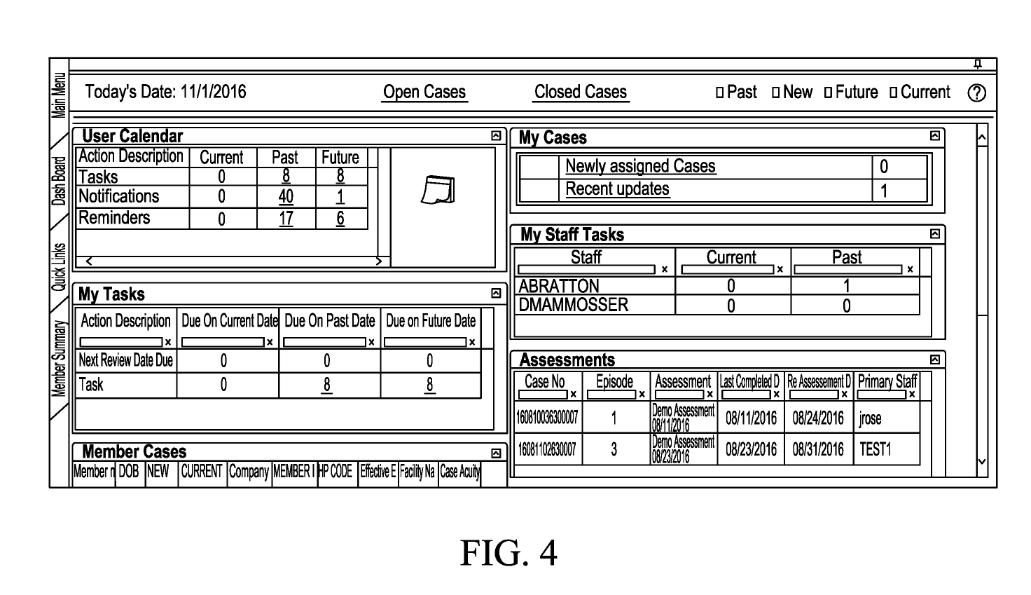

In one embodiment, one or more of the devices and systems, such as a data input worker, will can include a “Login” user interface (FIG. 3) from which a secured user (data input professional) can input an individual's health care data metrics. The data input worker (user) “Login” interface and a display interface for response to questions, which in one embodiment can be integrated, are generally configured to allow the input of queries and commands, as well as present the results of such command and queries.

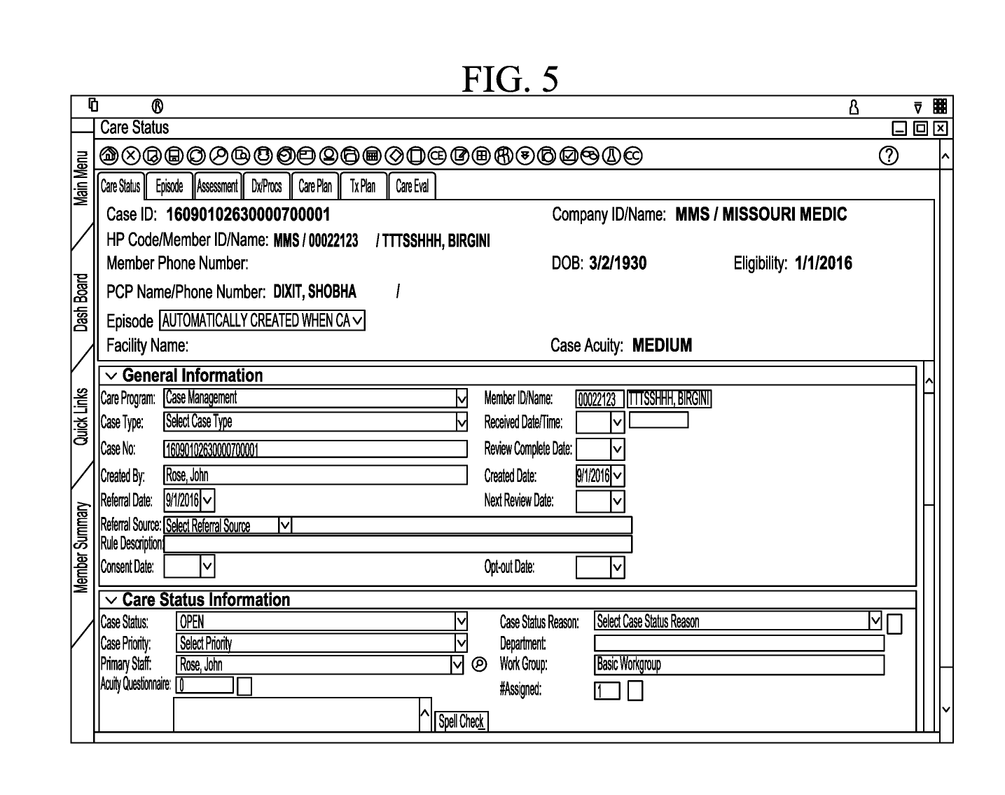

A subsequent data input interface as seen in FIG. 5, is provided where a particular individual patient's general information data may be input.

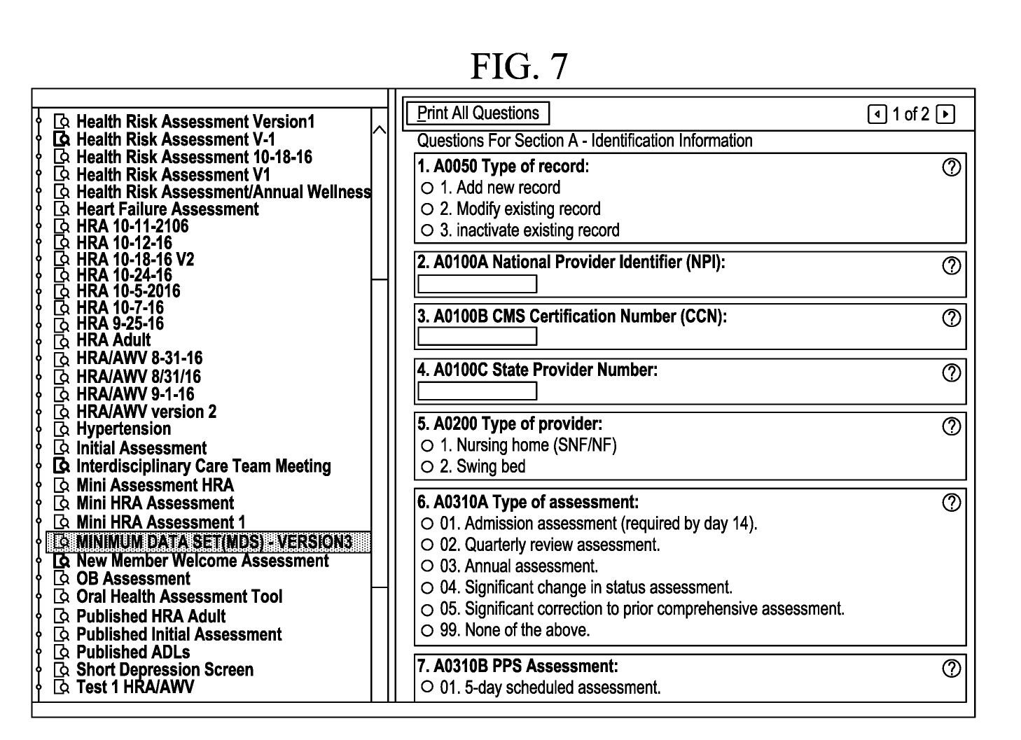

The computerized system will also include an interface for viewing and/or input of a minimum data set (MDS) relating to the individual, as shown in FIG. 7. This interface “dashboard” includes identifying information concerning the individual, such as insurance provider (Medicare, etc.) information, social security information, gender, and the like.

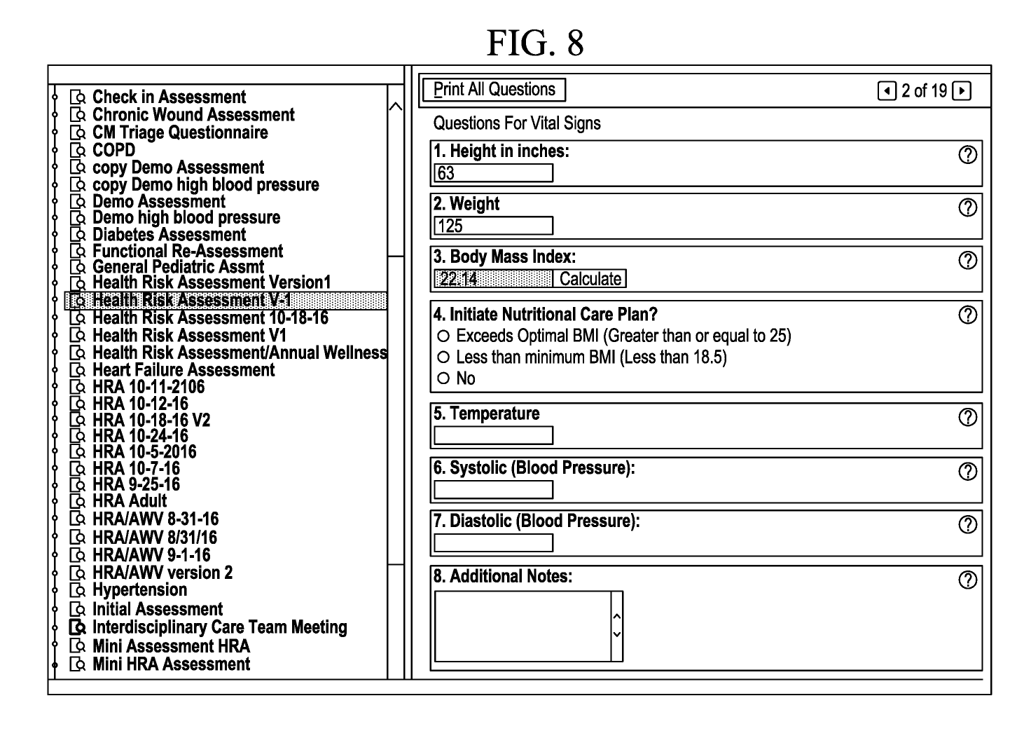

The computerized program also provides next for the input of data relating to the Health Risk Assessment Tool (HRAT), as described herein. At this interface page, the computerized system permits the input of background information concerning the patient, and permits this data to be securely transmitted to the central server of the system. This dashboard is shown at FIG. 8.

A computer interface (dashboard) for the Hospital Admission Risk Index information input page is provided in FIG. 8.

At FIG. 8, a computer interface (dashboard) is provided illustrating the Hospital Admission Risk Index interface. This interface permits input of data relating to the set of covariates (in some embodiments, the 22 covariates) of the Hospital Admission Risk index. Data input at this dashboard interface may also be in direct communication with the central server. This component of the computerized system will include a software program

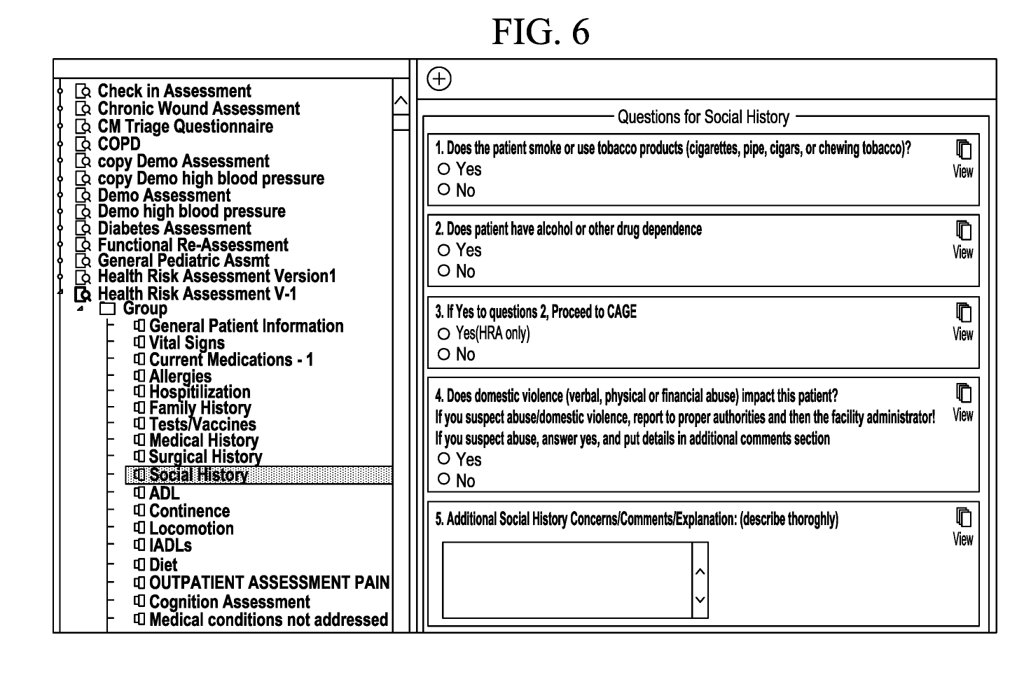

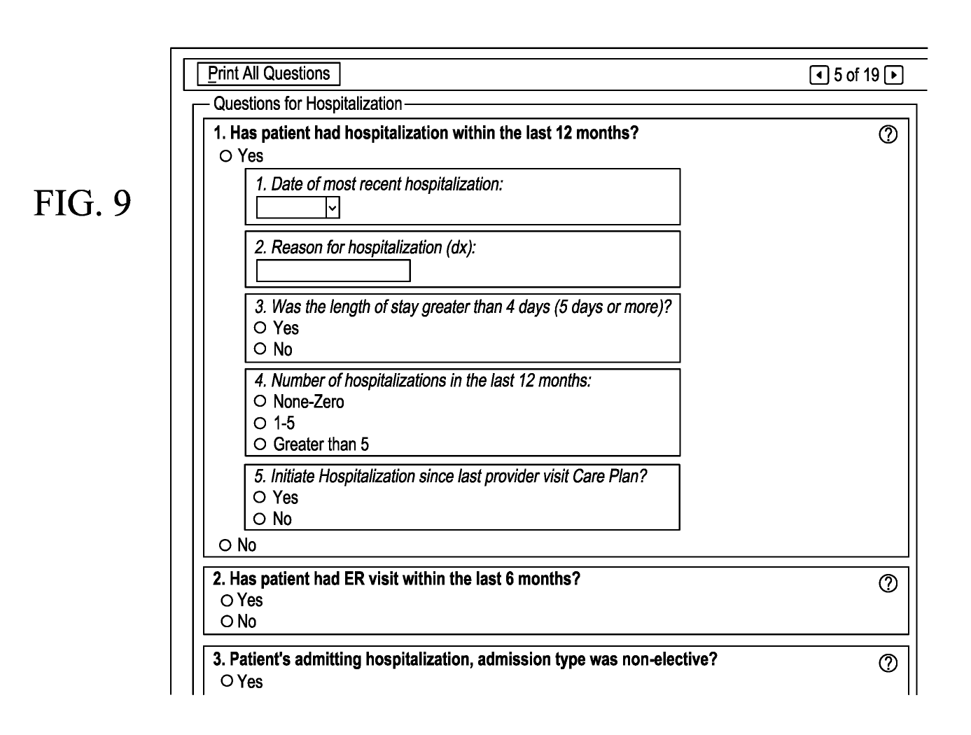

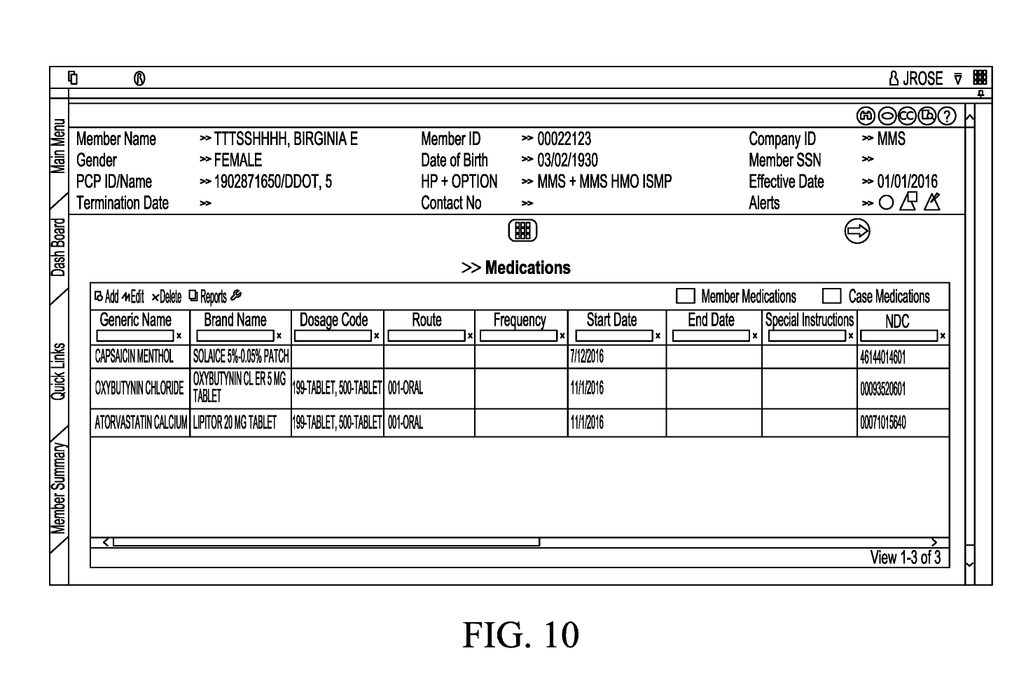

All data is communicated to the central server through input web-based user pages (FIG. 5, FIG. 6, FIG. 7, FIG. 8, FIG. 9, FIG. 10), and into a “Rules Engine” program (See FIG. 2). The “Rules Engine” program includes software that integrates evidence-based care protocols and transforms disparate data sources (e.g., enrollment, minimum data set, pharmacy, medical, health risk assessment, etc.) into actionable information.

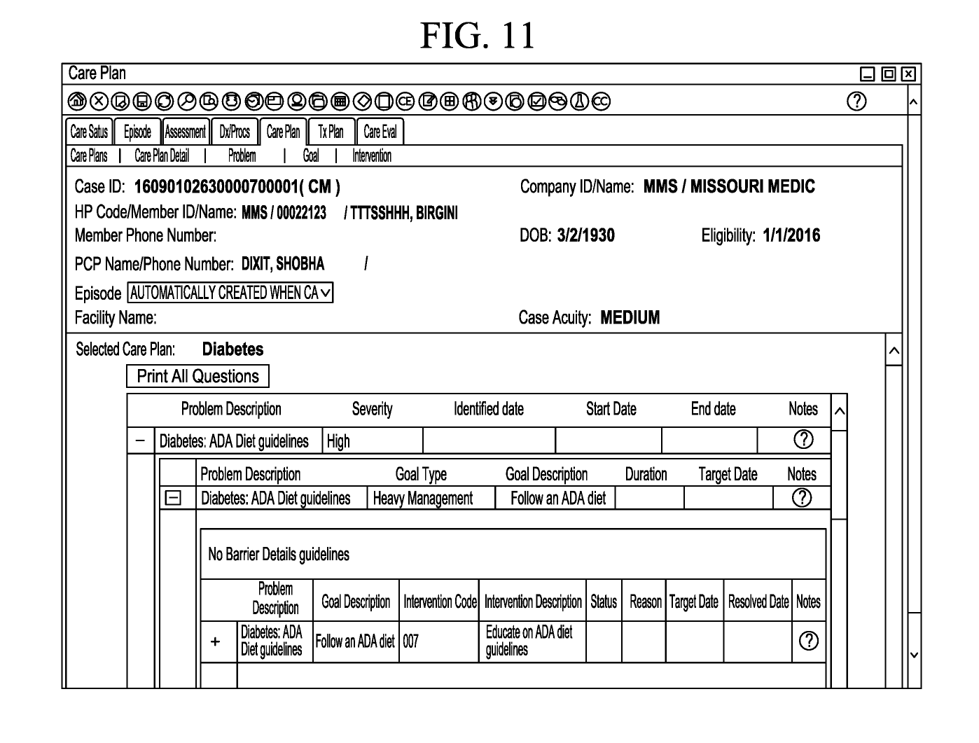

Computer software providing computer code that encodes the various functions and steps required to implement the Hospital Admission Risk Index methodology to carry out the individual resident scoring method provided here, is contained in the presently defined computer system. The computer software program is designed to assign a numerical point value to each answer to a proprietary set of “covariate” questions (in some embodiments, 22 “covariates”). The point score for a particular individual is then determined as a sum of these points for individual answers, and then weighted (normalized). The individual patient score may then be used to classify the patient into a high risk, medium risk or low risk group (See FIG. 1), where risk of admission and/or readmission to a hospital may be identified as part of the individual's risk group. The individual's risk group data may also be used in creating a plan of care for the patient. FIG. 11 presents a dashboard where “plan of care” is illustrated for an individual as part of the present computer based automated system.

### Example 5—Data Sets

The present example presents subsets of individual/resident data that may be used in the various applications of the present method and systems.

TABLE 4
Resident Data Pool Elements Subset - Hospital Admission Risk

| itm_id | itm_shrt_label | itm_type_cd |
|---|---|---|
| C0100 | BIMS: should resident interview be conducted | Code |
| C0200 | BIMS res interview: repetition of three words | Code |
| C0300A | BIMS res interview: able to report correct year | Code |
| C0300B | BIMS res interview: able to report correct month | Code |
| C0300C | BIMS res interview: can report correct day of week | Code |
| C0400A | BIMS res interview: able to recall “sock” | Code |
| C0400B | BIMS res interview: able to recall “blue” | Code |
| C0400C | BIMS res interview: able to recall “bed” | Code |
| C0500 | BIMS res interview: summary score | Number |
| C1000 | Cognitive skills for daily decision making | Code |
| H0100C | Appliances: ostomy | Checklist |
| H0400 | Bowel continence | Code |
| I0100 | Cancer (with or without metastasis) | Checklist |
| I0200 | Anemia | Checklist |
| I0600 | Heart failure | Checklist |
| I2100 | Septicemia | Checklist |
| I2400 | Viral hepatitis (includes type A, B, C, D, and E) | Checklist |
| I2900 | Diabetes mellitus (DM) | Checklist |
| I6200 | Asthma (COPD) or chronic lung disease | Checklist |
| J0100A | Pain: received scheduled pain med regimen | Code |
| J0100B | Pain: received PRN pain medications | Code |
| J0100C | Pain: received non-medication intervention | Code |
| J0200 | Should pain assessment interview be conducted | Code |
| J0300 | Res pain interview: presence | Code |
| J0400 | Res pain interview: frequency | Code |
| J0500A | Res pain interview: made it hard to sleep | Code |
| J0500B | Res pain interview: limited daily activities | Code |
| J0600A | Res pain interview: intensity rating scale | Number |
| J0600B | Res pain interview: verbal descriptor scale | Code |
| J0800A | Staff pain asmt: non-verbal sounds | Checklist |
| J0800B | Staff pain asmt: vocal complaints of pain | Checklist |
| J0800C | Staff pain asmt: facial expressions | Checklist |
| J0800D | Staff pain asmt: protective movements/postures | Checklist |
| J0800Z | Staff pain asmt: none of these signs observed | Checklist |
| J1400 | Prognosis: life expectancy of less than 6 months | Code |
| J1550D | Problem conditions: internal bleeding | Checklist |
| M0300A | Stage 1 pressure ulcers: number present | Number |
| M0210 | Resident has Stage 1 or higher pressure ulcers | Code |
| M0300B1 | Stage 2 pressure ulcers: number present | Number |
| M0300B2 | Stage 2 pressure ulcers: number at admit/reentry | Number |
| M0300C1 | Stage 3 pressure ulcers: number present | Number |
| M0300C2 | Stage 3 pressure ulcers: number at admit/reentry | Number |
| M0300B3 | Stage 2 pressure ulcers: date of oldest | Date |
| M0300D1 | Stage 4 pressure ulcers: number present | Number |
| M0300D2 | Stage 4 pressure ulcers: number at admit/reentry | Number |
| M0300E1 | Unstaged due to dressing: number present | Number |
| M0300E2 | Unstaged due to dressing: number at admit/reentry | Number |
| M0300F1 | Unstaged slough/eschar: number present | Number |
| M0300F2 | Unstaged slough/eschar: number at admit/reentry | Number |
| M0300G1 | Unstageable - deep tissue: number present | Number |
| M0300G2 | Unstageable - deep tissue: number at admit/reentry | Number |
| M0610A | Stage 3 or 4 pressure ulcer longest length | Number |
| M0610B | Stage 3 or 4 pressure ulcer width (same ulcer) | Number |
| M0700 | Tissue type for ulcer at most advanced stage | Code |
| M0800A | Worsened since prior asmt: Stage 2 pressure ulcers | Number |
| M0800B | Worsened since prior asmt: Stage 3 pressure ulcers | Number |
| M0800C | Worsened since prior asmt: Stage 4 pressure ulcers | Number |
| M0900A | Pressure ulcers on prior assessment | Code |
| M0900B | Healed pressure ulcers: Stage 2 | Number |
| M0900C | Healed pressure ulcers: Stage 3 | Number |
| M0900D | Healed pressure ulcers: Stage 4 | Number |
| M1040B | Other skin problems: diabetic foot ulcer(s) | Checklist |
| M1030 | Number of venous and arterial ulcers | Number |
| O0100A1 | Treatment: chemotherapy - while not resident | Checklist |
| O0100A2 | Treatment: chemotherapy - while resident | Checklist |
| O0100B1 | Treatment: radiation - while not resident | Checklist |
| O0100B2 | Treatment: radiation - while resident | Checklist |
| O0100C1 | Treatment: oxygen therapy - while not resident | Checklist |
| O0100C2 | Treatment: oxygen therapy - while resident | Checklist |
| O0100E1 | Treatment: tracheostomy care - while not resident | Checklist |
| O0100E2 | Treatment: tracheostomy care - while resident | Checklist |
| O0100H1 | Treatment: IV medications - while not resident | Checklist |
| O0100H2 | Treatment: IV medications - while resident | Checklist |
| O0100J1 | Treatment: dialysis - while not resident | Checklist |
| O0100J2 | Treatment: dialysis - while resident | Checklist |
| O0100K1 | Treatment: hospice care - while not resident | Checklist |
| O0100K2 | Treatment: hospice care - while resident | Checklist |
| G0110A1 | Bed mobility: self-performance | Code |
| G0110A2 | Bed mobility: support provided | Code |
| G0110B1 | Transfer: self-performance | Code |
| G0110B2 | Transfer: support provided | Code |
| G0110C1 | Walk in room: self-performance | Code |
| G0110C2 | Walk in room: support provided | Code |
| G0110D1 | Walk in corridor: self-performance | Code |
| G0110D2 | Walk in corridor: support provided | Code |
| G0110E1 | Locomotion on unit: self-performance | Code |
| G0110E2 | Locomotion on unit: support provided | Code |
| G0110F1 | Locomotion off unit: self-performance | Code |
| G0110F2 | Locomotion off unit: support provided | Code |
| G0110G1 | Dressing: self-performance | Code |
| G0110G2 | Dressing: support provided | Code |
| G0110H1 | Eating: self-performance | Code |
| G0110H2 | Eating: support provided | Code |
| G0110I1 | Toilet use: self-performance | Code |
| G0110I2 | Toilet use: support provided | Code |
| G0110J1 | Personal hygiene: self-performance | Code |
| G0110J2 | Personal hygiene: support provided | Code |
| G0120A | Bathing: self-performance | Code |
| G0120B | Bathing: support provided | Code |
| M0610C | Stage 3 or 4 pressure ulcer depth (same ulcer) | Number |
| N0350A | Insulin: insulin injections | Number |
| N0350B | Insulin: orders for insulin | Number |
| I6300 | Respiratory failure | Checklist |
| S0165E | Specialty services: On-Site Dialysis | Checklist |

TABLE 5
Continuum of Care Plan Elements

| itm_id | itm_shrt_label | itm_type_cd |
|---|---|---|
| I0300 | Atrial fibrillation and other dysrhythmias | Checklist |
| I0400 | Coronary artery disease (CAD) | Checklist |
| I0500 | Deep venous thrombosis (DVT), PE, or PTE | Checklist |
| I0600 | Heart failure | Checklist |
| I0700 | Hypertension | Checklist |
| I0900 | Peripheral vascular disease (PVD) or PAD | Checklist |
| I1200 | Gastroesophageal reflux disease (GERD) or ulcer | Checklist |
| I2000 | Pneumonia | Checklist |
| I2300 | Urinary tract infection (UTI) (LAST 30 DAYS) | Checklist |
| I2900 | Diabetes mellitus (DM) | Checklist |
| I3300 | Hyperlipidemia (e.g., hypercholesterolemia) | Checklist |
| I3800 | Osteoporosis | Checklist |
| I4200 | Alzheimer's disease | Checklist |
| I4500 | Cerebrovascular accident (CVA), TIA, or stroke | Checklist |
| I4800 | Non-Alzheimer's dementia | Checklist |
| I5800 | Depression (other than bipolar) | Checklist |
| I5900 | Manic depression (bipolar disease) | Checklist |
| I6000 | Schizophrenia | Checklist |
| I6200 | Asthma (COPD) or chronic lung disease | Checklist |
| J0100A | Pain: received scheduled pain med regimen | Code |
| J0100B | Pain: received PRN pain medications | Code |
| J0100C | Pain: received non-medication intervention | Code |
| J0200 | Should pain assessment interview be conducted | Code |
| J0300 | Res pain interview: presence | Code |
| J0400 | Res pain interview: frequency | Code |
| J0500A | Res pain interview: made it hard to sleep | Code |
| J0500B | Res pain interview: limited daily activities | Code |
| J0600A | Res pain interview: intensity rating scale | Number |
| J0600B | Res pain interview: verbal descriptor scale | Code |
| J0800A | Staff pain asmt: non-verbal sounds | Checklist |
| J0800B | Staff pain asmt: vocal complaints of pain | Checklist |
| J0800C | Staff pain asmt: facial expressions | Checklist |
| J0800D | Staff pain asmt: protective movements/postures | Checklist |
| J0800Z | Staff pain asmt: none of these signs observed | Checklist |
| J1550C | Problem conditions: dehydrated | Checklist |
| J1700A | Fall history: fall during month before admission | Code |
| J1700B | Fall history: fall 2-6 months before admission | Code |
| J1700C | Fall history: fracture from fall 6 month pre admit | Code |
| J1800 | Falls since admit/prior asmt: any falls | Code |
| J1900A | Falls since admit/prior asmt: no injury | Code |
| J1900B | Falls since admit/prior asmt: injury (not major) | Code |
| J1900C | Falls since admit/prior asmt: major injury | Code |
| K0200A | Height (in inches) | Number |
| K0200B | Weight (in pounds) | Number |
| L0200A | Dental: broken or loosely fitting denture | Checklist |
| L0200B | Dental: no natural teeth or tooth fragment(s) | Checklist |
| L0200C | Dental: abnormal mouth tissue | Checklist |
| L0200D | Dental: cavity or broken natural teeth | Checklist |
| L0200E | Dental: inflamed/bleeding gums or loose teeth | Checklist |
| L0200F | Dental: pain, discomfort, difficulty chewing | Checklist |
| L0200Z | Dental: none of the above | Checklist |
| M0100A | Risk determination: has ulcer, scar, or dressing | Checklist |
| M0150 | Is resident at risk of developing pressure ulcer | Code |
| M0300A | Stage 1 pressure ulcers: number present | Number |
| M0210 | Resident has Stage 1 or higher pressure ulcers | Code |
| M0300B1 | Stage 2 pressure ulcers: number present | Number |
| M0300B2 | Stage 2 pressure ulcers: number at admit/reentry | Number |
| M0300C1 | Stage 3 pressure ulcers: number present | Number |
| M0300C2 | Stage 3 pressure ulcers: number at admit/reentry | Number |
| M0300B3 | Stage 2 pressure ulcers: date of oldest | Date |
| M0300D1 | Stage 4 pressure ulcers: number present | Number |
| M0300D2 | Stage 4 pressure ulcers: number at admit/reentry | Number |
| M0300E1 | Unstaged due to dressing: number present | Number |
| M0300E2 | Unstaged due to dressing: number at admit/reentry | Number |
| M0300F1 | Unstaged slough/eschar: number present | Number |
| M0300F2 | Unstaged slough/eschar: number at admit/reentry | Number |
| M0300G1 | Unstageable - deep tissue: number present | Number |
| M0300G2 | Unstageable - deep tissue: number at admit/reentry | Number |
| M0610A | Stage 3 or 4 pressure ulcer longest length | Number |
| M0610B | Stage 3 or 4 pressure ulcer width (same ulcer) | Number |
| M0700 | Tissue type for ulcer at most advanced stage | Code |
| M0800A | Worsened since prior asmt: Stage 2 pressure ulcers | Number |
| M0800B | Worsened since prior asmt: Stage 3 pressure ulcers | Number |
| M0800C | Worsened since prior asmt: Stage 4 pressure ulcers | Number |
| M0900A | Pressure ulcers on prior assessment | Code |
| M0900B | Healed pressure ulcers: Stage 2 | Number |
| M0900C | Healed pressure ulcers: Stage 3 | Number |
| M0900D | Healed pressure ulcers: Stage 4 | Number |
| M1040A | Other skin problems: infection of the foot | Checklist |
| M1040B | Other skin problems: diabetic foot ulcer(s) | Checklist |
| M1040C | Other skin problems: other open lesion(s) on the foot | Checklist |
| M1040E | Other skin problems: surgical wound(s) | Checklist |
| M1040F | Other skin problems: burns (second or third degree) | Checklist |
| M1040Z | Other skin problems: none of the above | Checklist |
| M1030 | Number of venous and arterial ulcers | Number |
| M1200A | Skin/ulcer treat: pressure reduce device for chair | Checklist |
| M1200B | Skin/ulcer treat: pressure reducing device for bed | Checklist |
| M1200C | Skin/ulcer treat: turning/repositioning | Checklist |
| M1200D | Skin/ulcer treat: nutrition/hydration | Checklist |
| M1200E | Skin/ulcer treat: pressure ulcer care | Checklist |
| M1200F | Skin/ulcer treat: surgical wound care | Checklist |
| M1200G | Skin/ulcer treat: application of dressings | Checklist |
| M1200H | Skin/ulcer treat: apply ointments/medications | Checklist |
| M1200I | Skin/ulcer treat: apply dressings to feet | Checklist |
| M1200Z | Skin/ulcer treat: none of the above | Checklist |
| O0250A | Was influenza vaccine received | Code |
| O0250C | If influenza vaccine not received, state reason | Code |
| 00300A | Is pneumococcal vaccination up to date | Code |
| 00300B | If pneumococcal vacc not received, state reason | Code |
| V0200A02A | CAA-Cognitive loss/dementia: triggered | Checklist |
| V0200A02B | CAA-Cognitive loss/dementia: plan | Checklist |
| V0200A03A | CAA-Visual function: triggered | Checklist |
| V0200A03B | CAA-Visual function: plan | Checklist |
| V0200A04A | CAA-Communication: triggered | Checklist |
| V0200A04B | CAA-Communication: plan | Checklist |
| V0200A05A | CAA-ADL functional/rehab potential: triggered | Checklist |
| V0200A05B | CAA-ADL functional/rehab potential: plan | Checklist |
| V0200A06A | CAA-Urinary incont/indwell catheter: triggered | Checklist |
| V0200A06B | CAA-Urinary incont/indwell catheter: plan | Checklist |
| V0200A07A | CAA-Psychosocial well-being: triggered | Checklist |
| V0200A07B | CAA-Psychosocial well-being: plan | Checklist |
| V0200A08A | CAA-Mood state: triggered | Checklist |
| V0200A08B | CAA-Mood state: plan | Checklist |
| V0200A09A | CAA-Behavioral symptoms: triggered | Checklist |
| V0200A09B | CAA-Behavioral symptoms: plan | Checklist |
| V0200A10A | CAA-Activities: triggered | Checklist |
| V0200A10B | CAA-Activities: plan | Checklist |
| V0200A11A | CAA-Falls: triggered | Checklist |
| V0200A11B | CAA-Falls: plan | Checklist |
| V0200A12B | CAA-Nutritional status: plan | Checklist |
| V0200A13A | CAA-Feeding tubes: triggered | Checklist |
| V0200A13B | CAA-Feeding tubes: plan | Checklist |
| V0200A14A | CAA-Dehydration/fluid maintenance: triggered | Checklist |
| V0200A14B | CAA-Dehydration/fluid maintenance: plan | Checklist |
| V0200A15A | CAA-Dental care: triggered | Checklist |
| V0200A15B | CAA-Dental care: plan | Checklist |
| V0200A16A | CAA-Pressure ulcer: triggered | Checklist |
| V0200A16B | CAA-Pressure ulcer: plan | Checklist |
| V0200A17A | CAA-Psychotropic drug use: triggered | Checklist |
| V0200A17B | CAA-Psychotropic drug use: plan | Checklist |
| V0200A18A | CAA-Physical restraints: triggered | Checklist |
| V0200A18B | CAA-Physical restraints: plan | Checklist |
| V0200A19A | CAA-Pain: triggered | Checklist |
| V0200A19B | CAA-Pain: plan | Checklist |
| V0200A20A | CAA-Return to community referral: triggered | Checklist |
| V0200A20B | CAA-Return to community referral: plan | Checklist |
| V0200B2 | CAA-Assessment process signature date | Date |
| V0200C2 | CAA-Care planning signature date | Date |
| O0250B | Date influenza vaccine received. | Date |
| I6300 | Respiratory failure | Checklist |
| S1200A | Primary/secondary SMI dx: schizophrenia | Code |
| S1200B | Primary/secondary SMI dx: delusional disorder | Code |
| S1200C | Primary/secondary SMI dx: schizoaffective disorder | Code |
| S1200D | Primary/secondary SMI dx: psychotic disorder NOS | Code |
| S1200E | Primary/secondary SMI dx: bipolar disorder I | Code |
| S1200F | Primary/secondary SMI dx: bipolar disorder II | Code |
| S1200G | Primary/secondary SMI dx: cyclothymic disorder | Code |
| S1200H | Primary/secondary SMI dx: bipolar disorder NOS | Code |
| S1200I | Primary/secondary SMI dx: major depress recurrent | Code |
| S4500 | Substance Abuse: Alcoholic Drinks | Code |
| S4510A | Substance Abuse: Inhalants | Code |
| S4510B | Substance Abuse: Hallucinogens | Code |
| S4510C | Substance Abuse: Cocaine and Crack | Code |
| S4510D | Substance Abuse: Stimulants | Code |
| S4510E | Substance Abuse: Opiates | Code |
| S4510F | Substance Abuse: Cannabis | Code |
| S5000 | Number of New Pressure Ulcers | Number |
| S5005 | New Pressure Ulcer setting | Code |
| S5010A1 | Pressure ulcer 1 location | Code |
| S5010A2 | Pressure ulcer 1 status | Code |
| S5010B1 | Pressure ulcer 2 location | Code |
| S5010B2 | Pressure ulcer 2 status | Code |
| S5010C1 | Pressure ulcer 3 location | Code |
| S5010C2 | Pressure ulcer 3 status | Code |
| S5010D1 | Pressure ulcer 4 location | Code |
| S5010D2 | Pressure ulcer 4 status | Code |
| S5010E1 | Pressure ulcer 5 location | Code |
| S5010E2 | Pressure ulcer 5 status | Code |
| S5010F1 | Pressure ulcer 6 location | Code |
| S5010F2 | Pressure ulcer 6 status | Code |
| S5010G1 | Pressure ulcer 7 location | Code |
| S5010G2 | Pressure ulcer 7 status | Code |
| S5010H1 | Pressure ulcer 8 location | Code |
| S5010H2 | Pressure ulcer 8 status | Code |
| S5010I1 | Pressure ulcer 9 location | Code |
| S5010I2 | Pressure ulcer 9 status | Code |
| S0170A | Advanced directive: Guardian | Code |
| S0170B | Advanced directive: DPOA-HC | Code |
| S0170C | Advanced directive: Living will | Code |
| S0170D | Advanced directive: Do not resuscitate | Code |
| S0170E | Advanced directive: Do not hospitalize | Code |
| S0170F | Advanced directive: Do not intubate | Code |
| S0170G | Advanced directive: Feeding restrictions | Code |
| S0170H | Advanced directive: Other treatment restrictions | Code |
| S0170Z | Advanced directive: None of the above | Code |
| N0410A | Medication received: Days: antipsychotic | Number |
| N0410B | Medication received: Days: antianxiety | Number |
| N0410C | Medication received: Days: antidepressant | Number |
| N0410D | Medication received: Days: hypnotic | Number |
| N0410E | Medication received: Days: anticoagulant | Number |
| N0410F | Medication received: Days: antibiotic | Number |
| N0410G | Medication received: Days: diuretic | Number |

All references, including publications, patent applications, and patents, cited herein are hereby incorporated by reference to the same extent as if each reference were individually and specifically indicated to be incorporated by reference and were set forth in its entirety herein.

### BIBLIOGRAPHY

The following materials are specifically incorporated herein by reference.
1. Devons C A. Comprehensive geriatric assessment: Making the Most of the Aging Years. Curr Opin Clin Nutr Metab Care 2002; 5:19.
2. Stuck A E, Siu A L, Wieland G D, et al., Lancet 1993; 342: 1032
3. Segal M, Rollins E, Hodges K, Roozeboom M. Medicare-Medicaid Eligible Beneficiaries and Potentially Avoidable Hospitalizations. Medicare & Medicaid Research Review. 2014:4; 1
4. Segal M. CMS Policy Insight Brief. Dual Eligible Beneficiaries and Potentially Avoidable Hospitalizations. September 2011.
5. Xing J, Mukamel D B, Temkin-Greener H. J Am Geriatric Soc. 2013:61; 1900-08.
6. Jencks S F, Williams M V, Coleman E A. NEJM. 2009; 360:1418-28.
7. Smith, L, West S, Coots L, Ingber M, Reilly K, et al. Skilled Nursing Facility Readmission Measure: All-Cause Risk-Standardized Readmission Measure. CMS Contract No. HHSM-500-2013-130151 Task Order HHSM-500-T0001. March 2015.
8. Epstein-Lubow, G., Gaudiano, BA, Hinckley, M., Salloway, S., Miller, & I. W. (2010). Journal of the American Geriatrics Society, 58(2), 387-388.
9. Folstein M F, Folstein S E, McHugh P R., J Psychiatr Res 1975; 12:189-98.
10. Yaffe M J, Wolfson C, Weiss D, Lithwick M., J Elder Abuse Negl. 2008; 20 (3): 276-300.
11. Ewing, John A. “Detecting Alcoholism: JAMA 252: 1905-1907, 1984.
12. Rubenstein L Z, Harker J O, Salva A, Guigoz T, Vellas B. Screening for undernutrition in geriatric practice: developing the short-form mini nutritional assessment (MNA-SF). J Geront. 2001:56A; M366-377.
13. Yesavage J A, Brink T L, Rose T L, et al., J Psychiatr Res. 1982-83; 17(1):37-49.
14. Podsiadlo, D; Richardson, S (1991), Journal of the American Geriatrics Society 39 (2): 142-8.
15. Katz s., JAGS. 1983:31(12); 721-726.
16. Chalmers J, Johnson V, Tang, J, Titler M., Journal of Gerontological Nursing. 2004:30(11):5-12.
17. U.S. National Library of Medicine, Initials. (2009, May 20). 2009aa braden scale source information.
18. US Pub. 2015/0220699
19. US Pub. 2014/0278500
20. US Pub 2015/0169835

What is claimed is:

1. A system for assessing hospital readmission risk of a resident of a nursing facility, comprising:
obtaining data points for a group of selected covariate factors from the resident;
determining a cumulative raw covariate factor score for the resident, wherein a point value is assigned to each positive response to each of the selected covariate factors, and wherein the point value for each selected covariate factor is derived from a population of nursing facility residents with a single incidence of a hospitalization event without a recurrent hospitalization event;
preparing a normalized risk score for the resident to obtain a numerical score value of between 0 to 10,000; and,
identifying an individual risk score for hospital readmission within a defined period of time for the resident, wherein the resident is stratified into a high risk group, a medium risk group or a low risk group for hospital readmission based on the individual risk score.

2. The system of claim 1 wherein a High Risk Group is a normalized resident score of greater than 2,001 to 10,000, a Medium Risk Group is a normalized resident score of 1,100 to 2,000, and a Low Risk Group is a normalized resident score of 0 to 1,099. Rob—did these numbers change with you change in “platform” from 20 to 2000?

3. The system of claim 1 wherein the resident is a geriatric patient.

4. A system for determining a continuum of care plan for a nursing facility resident comprising:
determining a risk score for hospital readmission for the resident as defined in claim 1; and
providing a continuum of care plan for said resident, wherein
a resident having a low risk group score is administered a continuum of care plan that is consistent with routine resident care in a nursing facility;
a resident having a medium risk group score is administered a continuum of care plan that is modified from routine resident care in the nursing facility to accommodate the specific conditions identified in the selected covariant factors for the resident that increase the risk score above a low risk score; and
a resident having a high risk group score is administered a continuum of care that is modified from routine resident care in the nursing facility to include heightened resident monitoring and heightened continuum of care preventative measures for the selected covariant factors of the resident.

5. The method of claim 4 wherein heightened continuum preventative measures comprise:
providing a face-to-face and a follow-up call to the resident within about 30 days of initial hospitalization;
administering to the resident specific treatments identified in a hospital readmission prevention protocol;
administering a treatment to the resident specific for at least one disease identified in the resident;
administering an individualized care plan to the resident specific for the resident covariate factors; or
administering a chronic care improvement plan or treatment to the resident;

6. A computer program product for automated risk assessment of a nursing home resident for readmission to a hospital care facility comprising:
a computer program code means suitable for collecting health care data from a plurality of data sources, including a set of covariate elements of the nursing home resident;
a computer program code means suitable for inputting said data into a central computer capable of performing a health risk assessment for risk of hospital readmission of the resident, and executable computer code suitable for providing a calculation of a risk score for the resident, said central computer having a web-based application;
a computer program code means that upon execution is suitable for classifying the risk score for hospital readmission of the resident as high risk, medium risk or low risk; and
a computer program code means that upon execution is suitable for electronically transmitting the resident risk score classification to an identified recipient.

7. The computer program product of claim 6, wherein the computer program code means, when executed in the processor device, is further configured to stratify a total score identified for said resident using the resident health care data set, and to identify a risk group for the resident.

8. The computer program product of claim 6 wherein the health care data comprises a Resident Data Pool Elements Subset, a Continuum of Care Plan Elements Data Set or both.

9. The computer program product of claim 6, wherein the computer program code means, when executed in the processor device, is configured to link the resident identifying information with the resident medical record from a hospital electronic admission system of a health care facility, and further comprises an executable computer program code providing instructions for execution by the processor to receive a unique identifier from the hospital electronic admission system, and to establish the electronic medical record, and to securely and automatically transmit a risk assessment score for said resident to the identified recipient.

10. The computer program product of claim 8, wherein the computer program code means, when executed in the processor device, is configured to select a specialized continuum of care plan for the resident after discharge from a hospital facility.

11. The computer program product of claim 6, wherein the identified recipient of data for said resident is a nursing home.

12. The computer program product of claim 6, wherein the computer program code means, when executed in the processor device, is further configured to: receive a first time signal corresponding to an entry of the resident to a hospital facility; receive a second time signal corresponding to a completion of answer input for the resident to a set of Continuum of Care Plan Elements data, and provide a continuum of care plan for said resident.

13. The computer program product of claim 6, wherein the computer program code means, when executed in the processor device, will automatically stratify a resident into a high, medium or low risk group from said resident Hospital Admission Risk Index score.

14. The computer program product of claim 6, wherein the computer program code means when executed in the processor device is further configured to automatically upload any change in resident data.

15. The method of claim 6 wherein the resident is a geriatric resident.

16. A nursing home resident data analysis system for a computer having a memory, a central processing unit and a display, comprising:
A means for configuring said memory to store and perform a set of defined functions on a defined set of covariant elements as defined in Table 2;
A means for providing said central processing unit with data input into the memory; and
A means configured to relay a defined set of covariant elements into to the central processing unit.

17. The nursing home resident data analysis system of claim 16 wherein the display is a computer screen provided at an input portal.
